(19) United States
(12) Patent Application Publication
(10) Pub. No.: US 2026/0002585 A1
(43) Pub. Date: Jan. 1, 2026

(54) SYSTEM AND METHOD FOR DETECTING THE PRESENCE OF AS WELL AS FOR ANALYZING PARTICLES IN A FLOW OF LUBRICANT

(71) Applicant: AIRBUS HELICOPTERS TECHNIK GMBH, Calden (DE)

(72) Inventors: Jörg LITZBA, Fuldatal (DE); Jörg SCHMIEDEL, Kassel (DE); Farid AHMAD RAHIMI, Hamburg (DE)

(73) Assignee: AIRBUS HELICOPTERS TECHNIK GMBH, Calden (DE)

(21) Appl. No.: 19/111,398

(22) PCT Filed: Nov. 13, 2023
(86) PCT No.: PCT/EP2023/081574

§ 371 Date: Mar. 13, 2025

(30) Foreign Application Priority Data
Dec. 9, 2022 (DE) 10 2022 213 367.6

(51) Int. Cl. F16H 57/04; F16H 57/01; G01N 15/00; G01N 15/06; G01N 33/28

(52) U.S. Cl. CPC F16H 57/0405; F16H 57/01; F16H 57/0402; F16H 57/0434; G01N 15/0656; G01N 33/2858; F16H 2057/012; G01N 2015/0053

(57) ABSTRACT

A system for detecting the presence of as well as for analysing particles in a flow of lubricant, comprising a housing with a channel that guides a flow of lubricant and that fluidically connects a lubricant inlet and a lubricant outlet, further comprising a particle sensor which is configured to detect and to analyse particles present in the flow of lubricant, as well as a chip detector which is disposed downstream of the particle sensor and configured to locate and magnetically retain magnetisable particles present in the flow of lubricant. The invention also concerns a method for detecting the presence of as well as for analysing particles in a flow of lubricant. In addition, the invention concerns a gearbox comprising such a system.

### CROSS-REFERENCE TO RELATED APPLICATIONS

This application is the U.S. national phase of international application PCT/EP2023/081574 filed on Nov. 13, 2023, which claims priority to German patent application No. 102022213367.6 filed on Dec. 12, 2022, the disclosures of which are incorporated in their entireties by reference herein.

### TECHNICAL FIELD

The disclosure relates to a system and method for detecting the presence of as well as for analyzing particles in a flow of lubricant. The disclosure further relates to a gearbox comprising such a system.

### BACKGROUND

During the operation of aircraft gearboxes, metallic particles, in particular ferromagnetic particles, accumulate continuously due to wear, in particular due to abrasion; these can accumulate in the lubricant for the gearbox. The accumulation of ferromagnetic particles, such as iron particles, for example, can be an indicator of a malfunction or progressive wear in the gearbox, which sooner or later can lead to damage and ultimately to failure of the gearbox. The abrasion here is a measure of possible incipient damage to gearbox components, in particular of spacer components, such as cracking or wear, for example. Especially in the case of aircraft gearboxes, in particular helicopter gearboxes, which have to guarantee long-term safe operation, it is problematic that magnetic particles cannot easily be detected from the outside.

Magnetic plugs are known from the prior art, which essentially comprise a magnet and a holder for the magnet. The magnetic force of the magnetic plug traps small metallic particles or chips.

Detectors for chips are also known. These essentially consist of a magnet, a holder for it and a connection which can be electrically short-circuited by particles or chips in the area of the magnet. Such devices are used for locating and retaining magnetic or magnetisable particles and for the purpose of indicating their existence electrically. Detectors for chips are also known in English as “chip detectors”.

U.S. Pat. No. 5,179,346 A also discloses a device for detecting electrically conductive particles within a fluid. The device comprises a housing with a first and a second portion as well as a magnet with a first pole and a second pole which is positioned within the first portion of the housing, wherein the first and the second pole define a reference plane. A first electrode with a first contact surface is provided, which is positioned within the second portion of the housing and which extends in a direction generally away from the magnet. A second electrode having a second contact surface is also provided, positioned within the second portion of the housing and extending in a direction generally away from the magnet. The first and the second contact surfaces are spaced apart from each other and are each at an angle relative to the reference plane. The device further comprises means for sensing the electrical resistance between the first and the second contact electrodes and for generating a signal in response which is indicative of the presence of the electrically conductive particles.

### SUMMARY

The objective of the present disclosure is to provide an efficient and safe system and method for detecting the presence as well as for analyzing particles in a flow of lubricant. The objective of the present disclosure is to propose a gearbox with such a system.

For the purposes of the disclosure, the term “analysis of particles” should be understood to mean the determination of the size of the particles as well as the determination of an accumulation of the particles over time.

The objective is achieved by a system and method for detecting the presence of as well as for analyzing particles in a flow of lubricant, as well as by a gearbox.

In accordance with a first aspect of the disclosure, a system for detecting the presence of as well as for analyzing particles in a flow of lubricant comprises a housing with a channel that guides a flow of lubricant and that fluidically connects a lubricant inlet and a lubricant outlet, further comprising a particle sensor which is configured to detect and to analyse particles present in the flow of lubricant, as well as a chip detector which is arranged downstream of the particle sensor and configured to locate and magnetically retain magnetisable particles present in the flow of lubricant. The “system” should be understood to mean a sensor device which has different measuring systems and different measuring principles for detecting different measured variables and combining them with one another. By means of such a system, comparatively accurate wear monitoring can be carried out, since threshold values for measured variables can be defined more precisely. The disclosure makes it possible to assess the development of the quantity of magnetic particles in a gearbox as well as to make an assertion, in particular about the size and quantity of the particles present in the flow of lubricant. Lubricant is passed or conducted through the channel of the system, whereupon it is possible to detect, for example as a function of time, whether, which and how many particles are present in the flow of lubricant. Following this analysis by the particle sensor, at least the magnetic or magnetisable particles, in particular the ferromagnetic particles, are extracted from the lubricant by the chip detector and retained magnetically. To this end, the system is preferably intended to be arranged in a lubricant return line behind a component of the gearbox to be lubricated and/or cooled. Such a gearbox component may, for example, be a bearing element, a gearwheel or other gearbox components which are exposed to mechanical loads or which carry out a function pertaining to drive, in particular to transmit drive power, and are therefore to be lubricated and/or cooled.

The wording “downstream of the particle sensor” should be understood to mean that the flow of lubricant passes the particle sensor first, so that the flow of lubricant can be analysed with regard to particles present therein. Only then does the flow of lubricant pass the chip detector which, so to speak, filters magnetic or magnetisable particles out of the lubricant or extracts them magnetically.

The chip detector is a magnet which closes a contact and indicates when a specific quantity of magnetic particles which have been magnetically attracted out of the flow of lubricant and which adhere to it has been exceeded. The term “lubricant” should in particular be understood to mean engine oil or gearbox oil.

In one embodiment, the particle sensor is designed as an oil particle sensor. In particular, the particle sensor is used to measure ferromagnetic particles in the flow of lubricant. During the passage of lubricant through the particle sensor, a determination is made as to whether ferromagnetic or electrically conductive particles are present in the flow of lubricant. Ferromagnetic particles are detected, for example, via the change in the magnetic coupling between two coils. The particle sensor can generate an electric field, wherein the system can detect whether particles are present in the flow of lubricant by a change in the electric field. If electrically conductive particles are discerned or detected, the system can also identify what size they are and what quantity is passed through the channel of the housing over a specific period of time. In this sense, the particle sensor is configured to analyse electrically conductive particles present in the flow of lubricant at least with regard to size and quantity. The particle sensor therefore carries out particle monitoring. The particle sensor is preferably configured in a manner such that even non-metallic particles can be detected and analysed, provided that they are at least slightly electrically conductive.

The particle sensor interacts with particles in the channel in the flow of lubricant. The chip detector is disposed in or on the channel, wherein a section of the chip detector comes into direct contact with the lubricant within the channel in order to locate and magnetically retain magnetic or magnetisable particles in the flow of lubricant.

The channel is configured in a manner such that it can be used in a lubricant supply in a gearbox or a machine. To this end, the channel has, for example, a cross section which corresponds to a line or channel cross section of the lubricant supply for the gearbox or for the machine. The channel preferably has a circular shape in order to avoid turbulence in the flow of lubricant.

With the proposed system, the functions of the chip detector are integrated into a common overall system with the function of the particle sensor, i.e., particle monitoring. The system can be used to evaluate the criticality of the quantity of chips in relation to its development. At the same time, the function and efficiency of the chip detector can be ensured and monitored. Due to the combination of two different measuring systems and measuring principles in a common or integral unit, the installation space and masses which are required are also reduced. Directing the flow of lubricant or oil at the chip detector enables safer and more precise detection of the particles compared with the prior art, as well as better collection of the particles. The particle sensor and the chip detector may be connected to a monitoring unit, which may be integrated into a gearbox and/or vehicle controller.

Preferably, the chip detector can be detachably fastened to the housing by interlocking and/or force-fitting engagement. In other words, the chip detector is removably arranged on the housing so that the chip detector can be removed in order to remove adhering material. In accordance with one exemplary embodiment, the chip detector is screwed into the housing. The chip detector may also be preloaded in order to ensure a secure and positionally accurate fit on the housing.

Preferably, the particle sensor comprises a channel section which is integrated into the channel, wherein the channel section is arranged coaxially with respect to the lubricant inlet. The channel section forms a portion of the channel in the housing. The flow of lubricant is passed through the particle sensor, wherein the flow of lubricant passed through the channel section of the particle sensor is completely surrounded by the particle sensor in order to enable reliable analysis of the flow of lubricant. A coaxial disposition of the channel section of the particle sensor with respect to the lubricant inlet also ensures that the lubricant can be conducted as uniformly as possible, i.e., without turbulence, through the channel and the channel section of the particle sensor.

Alternatively, the channel section may also preferably have a coaxial offset with respect to the lubricant inlet, for example in order to be better capable of being integrated into the structure of the drive train.

More preferably, the longitudinal extent of the chip detector is arranged coaxially with respect to the lubricant inlet. Here again, a coaxial offset may be envisaged as a preferred alternative.

In accordance with one exemplary embodiment, the lubricant inlet, the channel section of the particle sensor and the longitudinal axis of the chip detector are arranged coaxially with respect to each other. By avoiding turbulence in the flow of lubricant, the chip detector can extract particles from the flow of lubricant and retain them magnetically in a better and more secure manner.

In accordance with an alternative exemplary embodiment, the particle sensor is arranged perpendicular to the lubricant inlet.

In accordance with one exemplary embodiment, the lubricant outlet is positioned obliquely with respect to the lubricant inlet. Preferably, the lubricant outlet is formed perpendicular to the lubricant inlet. The chip detector is preferably arranged in the bend, i.e., in a deflection point of the channel, between the lubricant inlet and the lubricant outlet.

The system for detecting the presence as well as for analyzing particles in a flow of lubricant is suitable for use in a gearbox for an aircraft, in particular for a helicopter gearbox. In particular, the gearbox is installed in a powertrain of a vehicle, in particular an aircraft. The term “aircraft” should be understood to mean a technical device which flies within the earth's atmosphere and is used to transport people or goods. In the context of this disclosure, an “aircraft” should be understood to mean an aeroplane or a helicopter.

In a second aspect of the disclosure, a method is provided for detecting the presence of as well as for analyzing particles in a flow of lubricant by means of a system for detecting the presence of as well as for analyzing particles in the flow of lubricant, comprising a housing with a channel that guides a flow of lubricant and that fluidically connects a lubricant inlet and a lubricant outlet, wherein particles present in the flow of lubricant are detected and analysed by means of a particle sensor, and wherein magnetisable particles present in the flow of lubricant are located and magnetically retained by means of a chip detector arranged downstream of the particle sensor.

In accordance with a third aspect of the disclosure, a gearbox for an aircraft, in particular for a helicopter gearbox, comprises a lubricant supply with a lubricant supply line for supplying a flow of lubricant to at least one gearbox component to be lubricated and a lubricant return line for returning a flow of lubricant, wherein a system for detecting the presence of as well as for analyzing particles in a flow of lubricant according to the first aspect of the disclosure is disposed in the lubricant return line.

The lubricant feed line and the lubricant return line are fluidically connected to a sump, in particular an oil sump or the like. The lubricant supply in the gearbox is used to lubricate and/or cool different components of the gearbox. The term “lubricant” should be understood to mean oil, in particular gear oil.

The system is arranged at the lowest point in the gearbox, where as many particles as possible are collected due to gravity and removed from the lubricant return line.

The above definitions and explanations of technical effects, advantages and advantageous embodiments of the system according to the disclosure in accordance with the first aspect of the disclosure also apply mutatis mutandis to the method according to the disclosure in accordance with the second aspect of the disclosure and to the gearbox according to the disclosure in accordance with the third aspect of the disclosure, and vice versa.

### BRIEF DESCRIPTION OF THE DRAWINGS

A preferred exemplary embodiment of the disclosure will be explained in more detail below with reference to the drawings, wherein:

FIG. 1 is a greatly simplified diagrammatic representation of a gearbox in accordance with the disclosure, comprising a system for detecting the presence as well as for analyzing particles in a flow of lubricant;

FIG. 2 shows a greatly simplified view of the system in accordance with the disclosure as shown in FIG. 1; and

FIG. 3 shows a greatly simplified longitudinal sectional view of the system in accordance with the disclosure according to FIG. 1 and FIG. 2.

### DETAILED DESCRIPTION

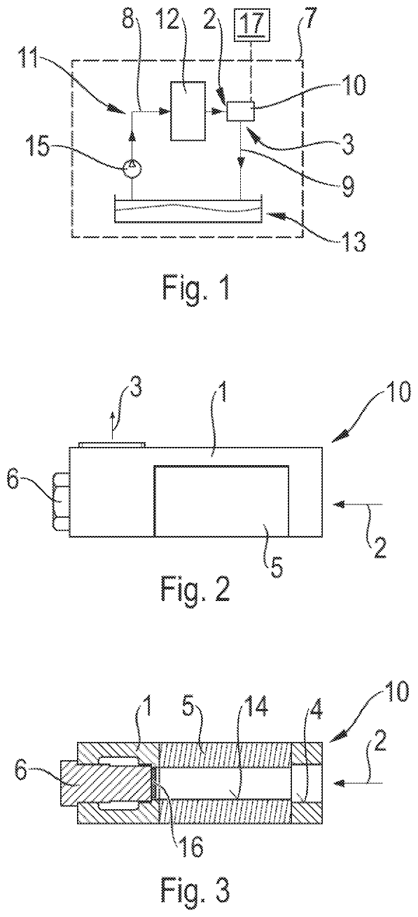

FIG. 1 is intended to show a gearbox 7 designed as a helicopter gearbox in a greatly simplified manner. The gearbox 7 is operatively connected for the purposes of drive to a drive unit—not shown here—in order to drive the helicopter. The gearbox 7 comprises a lubricant supply 11 for lubricating and cooling gearbox components 12. Such a gearbox component 12 of the gearbox 7 is represented here in a greatly simplified manner by a rectangle. The lubricant supply 11 has a lubricant supply line 8 for supplying lubricant to the gearbox component 12 to be lubricated and a lubricant return line 9 for returning the lubricant to an oil sump 13. The lubricant, here in the form of gear oil, is delivered by a pump 15. Disposed in the lubricant return line 9, i.e., in the flow of lubricant downstream of the gearbox component 12, is a system 10 in accordance with the disclosure for detecting the presence of as well as for analyzing particles in a flow of lubricant, which is configured to detect particles in the flow of lubricant and to analyse them in order to draw conclusions about the wear behaviour of the gearbox 7, in particular of the gearbox component 12. The system 10 is shown in FIG. 2 and FIG. 3 and will be described in more detail below.

Said system 10 is designed and configured to carry out a method in accordance with the disclosure for detecting the presence of as well as for analyzing particles in a flow of lubricant as follows. The system 10 communicates with a monitoring unit 17, which outputs a warning signal in the event that a threshold value is exceeded. The warning signal can be output in any desired manner and with known means.

The system 10 is a sensor device which has a plurality of measuring systems with different measuring principles for detecting different measured variables. According to FIG. 2 and FIG. 3, the system 10 comprises a housing 1 with a channel 4 which guides a flow of lubricant and fluidically connects a lubricant inlet 2 and a lubricant outlet 3. The lubricant outlet 3 is substantially perpendicular in configuration to the lubricant inlet 2 (cf. FIG. 2), wherein the lubricant inlet 2 and the lubricant outlet 3 in FIG. 2 and FIG. 3 are symbolically represented just by arrows. At the same time, the arrows indicate the direction of flow of the lubricant—not shown here—through the channel 4.

The system 10 also comprises a particle sensor 5 as well as a chip detector 6. The particle sensor 5 is integrated into the housing 1, wherein the chip detector 6 is disposed so as to be removable from the housing 1. The chip detector 6 here is screwed into the housing 1.

The particle sensor 5 is configured to detect and to analyse particles present in the flow of lubricant. The particle sensor 5 is capable of detecting magnetic, magnetisable as well as other particles in the flow of lubricant and to analyse them with respect to their type, size and quantity. As a result, a change in the quantity and composition of the particles can be determined over the useful life of the gearbox component 12. The particle sensor 5 comprises a channel section 14, which is integrated into the channel 4. According to FIG. 3, the channel section 14 is disposed coaxially with respect to the lubricant inlet 2, by way of example.

The chip detector 6 disposed downstream of the particle sensor 5 is configured to locate and magnetically retain the magnetic or magnetisable particles present in the flow of lubricant. The chip detector 6 accordingly extracts magnetic or magnetisable particles from the flow of lubricant passing through the chip detector 6 before the lubricant is conveyed out of the housing 1 and back into the oil sump 13 via the pump 15. An oil filter-not shown here-which can filter out non-magnetic or non-magnetisable particles, in particular dirt, can additionally be disposed upstream of the chip detector 6 and downstream of the pump 15. The longitudinal extent of the chip detector 6 is also disposed coaxially with respect to the lubricant inlet 2. A magnetic section 16 of the chip detector 6 is disposed, for example, in the region of a deflection point of the channel 4, where the lubricant coming from the lubricant inlet 2 and the particle sensor 5 is deflected and guided to the lubricant outlet 3. The deflection point of the channel 4 is not shown here in detail.

The particle sensor 5 and the chip detector 6 communicate with the monitoring unit 17, whereupon if a threshold value is exceeded, an appropriate signal is sent to the monitoring unit 17, which indicates this accordingly.

Clearly, it is conceivable for a plurality of such systems 10 to be disposed in a gearbox 7 for an aircraft. In particular, two systems 10 in accordance with the disclosure may be disposed in a gearbox 7. However, disposing more than two systems 10 in accordance with the disclosure in a gearbox 7 is also conceivable. The disclosure is therefore not limited to just a single system 10 in a gearbox 7. It is further conceivable that the wear of a plurality of gearbox components or component groups could be monitored by a system 10.

### LIST OF REFERENCE NUMERALS

1 housing
2 lubricant inlet
3 lubricant outlet
4 channel
5 particle sensor
6 chip detector
7 gearbox
8 lubricant supply line
9 lubricant return line
10 system
11 lubricant supply
12 gearbox component
13 oil sump
14 channel section
15 pump
16 magnetic section of the chip detector
17 monitoring unit

1. A system for detecting the presence of as well as for analysing particles in a flow of lubricant, comprising a housing with a channel that guides a flow of lubricant and that fluidically connects a lubricant inlet and a lubricant outlet, further comprising a particle sensor which is configured to detect and to analyse particles present in the flow of lubricant, as well as a chip detector which is arranged downstream of the particle sensor and configured to locate and magnetically retain magnetisable particles present in the flow of lubricant.

2. The system according to claim 1, wherein the chip detector is releasably attachable to the housing by interlocking and/or force-fitting engagement.

3. The system according to claim 2, wherein the chip detector is screwed into the housing.

4. The system according to claim 1, wherein the particle sensor is configured to analyse electrically conductive particles present in the flow of lubricant, at least in respect of size and quantity.

5. The system according to claim 1, wherein the particle sensor comprises a channel section which is integrated into the channel, wherein the channel section is arranged coaxially with respect to the lubricant inlet.

6. The system according to claim 1, wherein the longitudinal extension of the chip detector is arranged coaxially with respect to the lubricant inlet.

7. The system according to claim 1, wherein the lubricant outlet is positioned obliquely with respect to the lubricant inlet.

8. A method for detecting the presence of as well as for analysing particles in a flow of lubricant by means of a system for detecting the presence as well as for analysing particles in the flow of lubricant, comprising a housing with a channel that guides a flow of lubricant and that fluidically connects a lubricant inlet and a lubricant outlet, wherein particles present in the flow of lubricant are detected and analysed by means of a particle sensor (5), and wherein magnetisable particles present in the flow of lubricant are located and magnetically retained by means of a chip detector arranged downstream of the particle sensor.

9. Use of a system for detecting the presence of as well as for analysing particles in a flow of lubricant according to claim 1 in a gearbox for an aircraft.

10. A gearbox for an aircraft, in particular for a helicopter gearbox, comprising a lubricant supply with a lubricant supply line for supplying a flow of lubricant to at least one gearbox component to be lubricated and a lubricant return line for returning a flow of lubricant, wherein a system for detecting the presence of as well as for analysing particles in a flow of lubricant according to claim 1 is arranged in the lubricant return line.
